(19) United States
(12) Patent Application Publication
(10) Pub. No.: US 2023/0212824 A1
(43) Pub. Date: Jul. 6, 2023

(54) METHOD AND APPARATUS FOR CONDITIONING OF CARDBOARD

(71) Applicant: Highcon Systems Ltd., Yavne (IL)

(72) Inventors: Nuriel AMIR, Yokneam Illit (IL); Michael KARP, Rosh Ha-Ain (IL); Or BEN-DAVID, Ramat-Gan (IL)

(21) Appl. No.: 18/105,271

(22) Filed: Feb. 3, 2023

Related U.S. Application Data
(63) Continuation-in-part of application No. PCT/IL2021/050941, filed on Aug. 3, 2021.
(60) Provisional application No. 63/061,382, filed on Aug. 5, 2020.

(51) Int. Cl. D21H 25/04; A61L 9/015

(52) U.S. Cl. CPC D21H 25/04; A61L 9/015; A61L 2209/14; A61L 2209/16

(57) ABSTRACT

A method and apparatus are disclosed for conditioning a corrugated cardboard stack by causing air to flow through flutes of the stack from an air intake end to an air discharge end of the stack, air moved by an air moving device being confined to flow only, or at least predominantly, through the flutes.

### RELATED APPLICATIONS

This application is a continuation-in-part of PCT/IL2021/050941, filed 03 Aug. 2021, which claims the benefit of USSN 63/061382, filed 05 Aug. 2020. The disclosures of both of the aforesaid applications are incorporated herein by reference in their entireties.

### FIELD OF THE INVENTION

The invention relates to treatment of cardboard, in particular corrugated cardboard, for instance following processing in a conversion apparatus, in order to remove burn fumes and odor caused by laser cutting of the cardboard.

### BACKGROUND OF THE INVENTION

The use of laser beams to cut and perforate cardboard (as used in conversion machines such as Highcon Euclid or Highcon Beam available from Highcon Systems Ltd. in Yavne, Israel) creates undesired fumes and odor due to the burning of the cardboard during the process. To reduce the odor, it is common practice to leave the cardboard to ventilate in free air. Even when fans or blowers are used to cause air circulation, it can take a day or more for the smell to be reduced to an acceptable level. Such a method is therefore not efficient, in particular for corrugated cardboard.

An alternative method is described in U.S. Pat. US 10,179,378, which describes removing the smell by heating the cardboard to a temperature as high as 150-260° C. using laser light or in an oven. Such a method is wasteful of energy and dries out the cardboard.

### OBJECT OF THE INVENTION

The invention seeks to provide a method and apparatus for conditioning cardboard, by removing fumes and odor more effectively.

### SUMMARY OF THE INVENTION

According to a first aspect of the invention, there is provided a method of conditioning a corrugated cardboard stack, which comprises causing air to flow through flutes of the stack from an air intake end to an air discharge end of the stack by confining air moved by an air moving device to flow only, or at least predominantly, through the flutes. The term “flutes” is used herein to describe the flow channels defined by the corrugations.

After treatment in a conversion machine, cardboard is commonly in the form of individual sheets and the cardboard stack may typically comprise several separate sheets placed one over the other. However, the term “cardboard stack” as used herein is also intended to include cardboard that is coiled into a roll having overlying, or stacked, layers rather than individual sheets and in the ensuing description all references to a stack should be regarded as including a roll.

The invention is predicated on the realisation that, when cardboard is exposed to fumes, such as the fumes that are created during laser cutting, they tend to permeate into the flutes defined by the corrugations. If, as has been the common practice in the past, a corrugated cardboard stack is merely placed in a room in which air circulates, the air flows predominantly around the outer surface of the stack, because it encounters far less resistance that when flowing through the flutes. By guiding the air to ensure that it flows through the flutes, in preference to flowing around the stack, the invention ensures more effective and rapid removal of the fumes and odor.

In some embodiments of the invention, the air moving device may comprise an air extractor connected to the air discharge end of the stack to place the air discharge end under sub-atmospheric pressure and thereby to draw ambient air into the flutes at the air intake end.

Alternatively, or additionally, the air moving device may comprise a fan or blower connected to the air intake end of the stack to raise the air pressure at the air intake end above ambient atmospheric pressure and thereby blow air into the flutes.

In some embodiments, the same air moving device may serve both to reduce the air pressure at the discharge end of the stack and to increase the air pressure at the air intake end, thereby causing air to recirculate through the stack. In this case, to prevent the extracted fumes from recirculating, the recirculated air may be filtered.

As well as removing the smell, in order to preserve certain characteristics of the cardboard, especially its crease readiness quality (i.e. its ability to crease without cracking), it is necessary to control its moisture content. For this reason, after removal of the smell, it is common practice to condition the cardboard by storing it in a moisture-controlled and temperature-controlled room, typically at a temperature in the range of 18-25° C. and a relative humidity of 50-60%RH, for a few days.

In some embodiments, the temperature of the recirculating air is controlled by heating and/or cooling the air to remain within a predetermined range. If desired, the humidity of the recirculating air may additionally, or alternatively, be controlled by a humidifier to remain within a predetermined range.

According to as second aspect of the invention, there is provided an apparatus for conditioning a corrugated cardboard stack, which comprises an air moving device and a shroud to surround the stack so as to confine air moved by the device to flow only, or at least predominantly, through flutes of the cardboard in the stack, from an air intake end to an air discharge end of the stack.

In some embodiments, the shroud may include an element made of a flexible material that is urged to seal against the sides of the stack by an air pressure differential generated by the air moving device.

The air moving device may, in some embodiments, comprise an air extractor connected to the air discharge end of the stack to place the air discharge end under sub-atmospheric pressure and thereby to draw ambient air into the flutes at the air intake end.

Alternatively, or additionally, the air moving device may comprise a fan or blower connected to the air intake end of the stack to raise the air pressure at the air intake end above ambient atmospheric pressure and thereby blow air into the flutes.

The same air moving device may, in some embodiments, serve both to reduce the air pressure at the discharge end of the stack and to increase the air pressure at the air intake end, thereby creating an air recirculation circuit passing through the flutes of the cardboard in the stack. In such an embodiments, the recirculation circuit may include a filter, for example containing activated carbon.

For improved conditioning of the cardboard, the recirculation circuit may additionally include at least one of
(i) a heater or cooler for maintaining the temperature of the recirculating air within a predetermined range,
(ii) a humidifier for maintaining the humidity of the recirculating air within a predetermined range,
(iii) an ozoniser for introducing ozone into the recirculating air, and
(iv) and atomiser for introducing airborne material into the recirculating air.

### BRIEF DESCRIPTION OF THE DRAWINGS

The invention will now be described further, by way of example, with reference to the accompanying drawings, in which:

FIG. 1 shows a stack of cardboard sheets and arrows to demonstrate the desired air flow through the stack,

FIG. 2 shows a perspective view of a first embodiment of an apparatus of the invention,

FIG. 3 is a plan view of a second embodiment of an apparatus of the invention,

FIG. 4 is a plan view of a further embodiment of the invention,

FIG. 5 is a photograph of an embodiment of the invention in which a shroud was not employed as part of the apparatus, and

FIG. 6 is a photograph of an embodiment of the invention in which a shroud was employed as part of the apparatus.

### DETAILED DESCRIPTION OF THE DRAWINGS

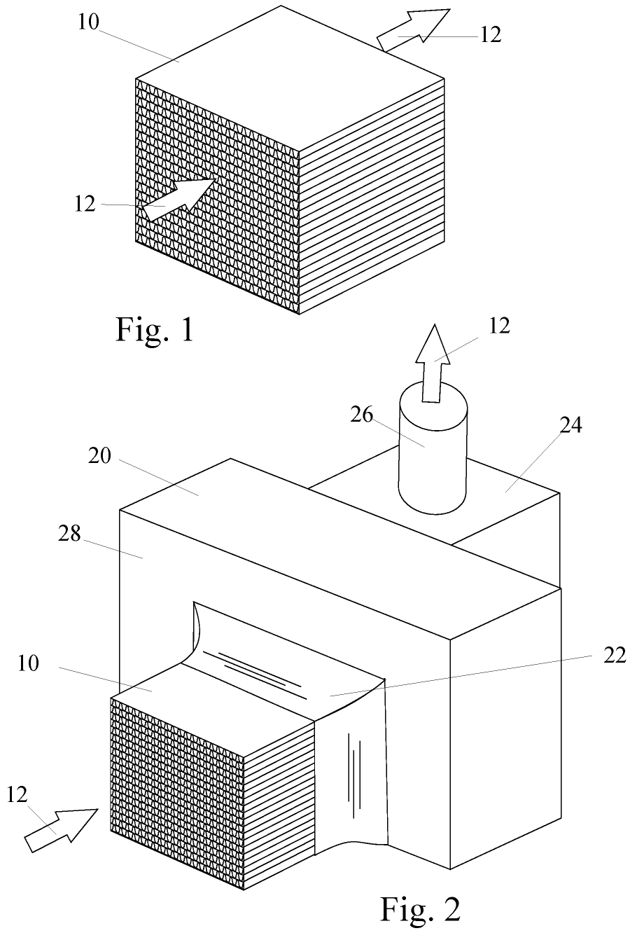

FIG. 1 shows a stack 10 made up of cardboard sheets that have all been stacked with the same orientation, that is to say with all the corrugations lying parallel to one another. Each sheet may have been pre-treated in a conversion machine, such as a Highcon Euclid or a Highcon Beam machine. The cutting of cardboard using a laser by such machines generates fumes that create an unpleasant odor. While FIG. 1 shows a cubic stack of individual sheets, as previously mentioned, the invention is equally applicable to removing odor from a roll of cardboard, which may have acquired an odor through improper storage or exposure to fumes.

In order to reduce the smell, the present invention proposes flowing air in the direction indicated by the arrows 12 in FIG. 1, that it to say with the air flowing through flutes defined by the corrugations of the cardboard, rather than around the stack 10. In this way, most of the cardboard is placed in the immediate vicinity of the air flow, being spaced from the air flow preferably by less than a few millimetres, or even less than one millimetre.

If a cardboard stack is merely left to ventilate, any air flow that takes place through the flutes of the stack is almost insignificant. This is because, even if the stack is placed in air stream, the flow resistance around the stack is much less that the flow resistance through the stack. To achieve the desired air flow through the stack, embodiments of the invention therefore confine the air flow, so as to prevent it as much as practicable from flowing around the outside of the stack.

In FIG. 2, ambient air is sucked through the stack 10 in the direction of arrows 12 by means of an air extractor 24. The intake end of the extractor 24 is connected to a plenum 20. The plenum 20 has on its front side 28 facing the stack 10, a mouth, which may be covered by a grille, against which the stack 10 is placed. A shroud 22 in the form of a skirt of a flexible material is attached to front side 28 of the plenum 20 and surrounds the mouth and the stack 10.

In use, the stack 10 is transported, for example by means of a forklift truck, and placed in front of the mouth in the front side 28 of the plenum 20 and the shroud 22 is loosely placed over the sides of the stack 10. When the extractor 24 is then operated, the low pressure created in the plenum 20 sucks the shroud into intimate contact with the stack 10 and the against the grille, so that no air can flow around the stack 10. Instead, the moved air follows the path indicated by the arrows 12 and flows through the flutes defined by the corrugations int the stack into the plenum 20, being then discharged to atmosphere through an exhaust duct 26 of the extractor 24.

The use of a flexible, air-impermeable material, as a shroud 22 has been found effective as it can form a good seal against the stack 10, even if the cardboard sheets in the stack 10 are not perfectly aligned. It should be clear to the person skilled in the art that other means may be employed to shroud the stack in order to prevent or minimise the flow of air around the stack 10. For example, slidable shutter plates may be mounted on the front side 28 of the plenum, or the mouth may be surround by an inflatable bladder or a ring of compressible, closed cell, foam.

In the embodiment of FIG. 2 there is no air recirculation, and the apparatus relies only on drawing ambient air into the stack 10 and discharging the air, together with any scavenged fumes, into the ambient atmosphere through the exhaust 26. If desired, a filter may be placed in the plenum 20 or in the exhaust duct 26 to reduce pollution of the ambient air.

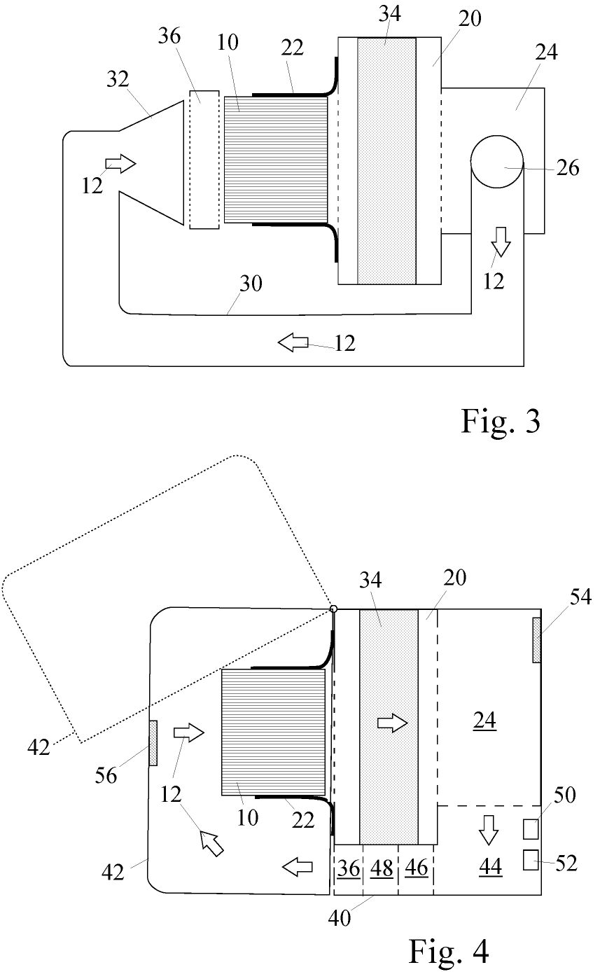

The embodiment of FIG. 3 is a modification of the embodiment of FIG. 2 and, in order to avoid repetition, like parts have been reallocated the same reference numerals. The essential difference between the embodiment of FIG. 3 and that of FIG. 2 is that the air discharged through the exhaust duct 26 of the extractor 24 is recirculated, via an air duct 30 and a horn 32, back to the air intake side of the stack 10 so that much of the air is recirculated and only a small proportion escapes to the ambient atmosphere through the gap between the horn 32 and the stack 10. By increasing the pressure differential across the stack, the embodiment of FIG. 3 allows the air flow rate to be nearly doubled and this has been found to improve odor removal even in the absence of a volatile organic compound (VOC) filter.

FIG. 3 also shows that a VOC filter 34 may be mounted in the plenum, to neutralise the fumes scavenged from the stack 10. The filter may for example be an activated carbon filter. As previously mentioned, such a filter may also be included in the embodiment of FIG. 2.

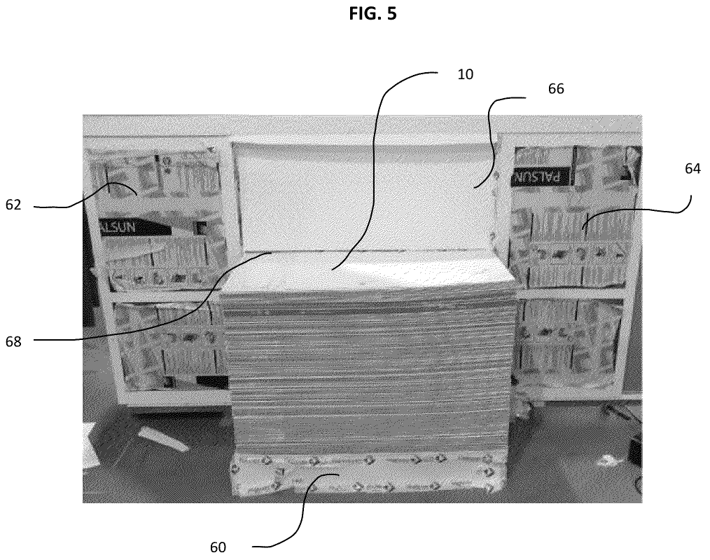

FIG. 3 also shows an ozoniser 36 is provided to introduce ozone into the air entering the stack 10. The ozone, which may be generated for example by an ultraviolet lamp, has a sterilising effect and also assists in regeneration of the activated carbon filter 34. Ozone may alternatively be generated by commercially available ozonisers, as available for example by Ecozone Technologies in Hasharon Industrial Park, Kadima, Israel (https://ecozone-technologies.com/). Various walls have been shown in FIGS. 3 and 4 in dotted lines, this being to indicate that air is allowed to pass through them.

In the embodiment of FIG. 4, like parts have again been allocated the same reference numerals. As in the embodiment of FIG. 3, the air in this embodiment is recirculated but instead of flowing through an air duct, it flows through a housing comprises of a stationary part 40 and a pivotable cover 42, show in solid lines in its closed position and in dotted lines in an open position. In addition to an ozoniser 36, as previously described, various devices are arranged within the housing 40, 42.

Reference numeral 44 represents temperature control equipment, that may comprise a heater and/or a cooler, for maintaining the temperature of the recirculated air within a predetermined desired range. A humidifier 46 is provided to regulate the relative humidity of the air.

In FIG. 4, an atomiser 48 is also provided to spray airborne material into the air. The sprayed material may be any desired treatment solution, for example it may be a perfume or an anti-bacterial compound.

FIG. 4 also shows that sensors 50 and 52 may be mounted in the housing to monitor desired parameters of the recirculating air, such as its temperature and relative humidity, the output of the sensor being provided to a control circuit that controls the operation of the heater/cooler and humidifier.

It is additionally possible to use as a sensor an ‘artificial nose’, such as Cyranose 320 commercially available from Sensigent in Baldwin Park, CA, USA or any other suitable smoke detector) in order to “smell” the level of fumes or odor removal which may also allow for automatic stopping of the conditioning operation when the level of odor reduction (or level of moisture) is achieved.

The embodiment of FIG. 4 offers the advantage that when the cover 42 is open, there is unhindered access to the mouth of the plenum 20. A stack 10 can therefore readily be placed in front of the plenum 20 and the shroud 22 deployed to seal around the stack 10. When next the cover 42 is pivoted into its closed position, a closed recirculation path, represented by the arrows 12 in the drawing, is created and clean air at a desired temperature and relative humidity can be circulated through the flutes of the stack 10 to remove any odor and also condition the cardboard.

If the pre-processed cardboard sheets have cuts in them, when they are stacked they may create paths for air to enter or exit from the stack, other than through its intake and discharge ends. If a shroud of flexible material is used, it may block air flow through these additional paths to ensure that air flow through the entire length of the flutes.

FIG. 4 also shows that an exhaust port 54 may be provided in the stationary part 40 of the housing. The port may be kept closed during air recirculation and only opened to vent the housing and discharge accumulated VOC’s to atmosphere between operating cycles. Alternatively, a proportion of the recirculating air may be discharged during operation and replenished with clear air, whereupon an intake port 56 containing a one-way valve may be provided in the cover 42.

The inventors have found that a suitable temperature range for heating the recirculating air is 30° - 80° C., preferably 40° - 60° C. An air flow through flutes at a speed of 0.3 - 15 m/s, preferably 2.5 - 10 m/s, for a period of only 3 - 15 min was found to suffice for effective removal of odors.

To compensate for paper moisture loss due to the heating, the humidifier may be controlled to maintain the relative humidity of the circulating air at 40-80%, and more preferably 50-70%.

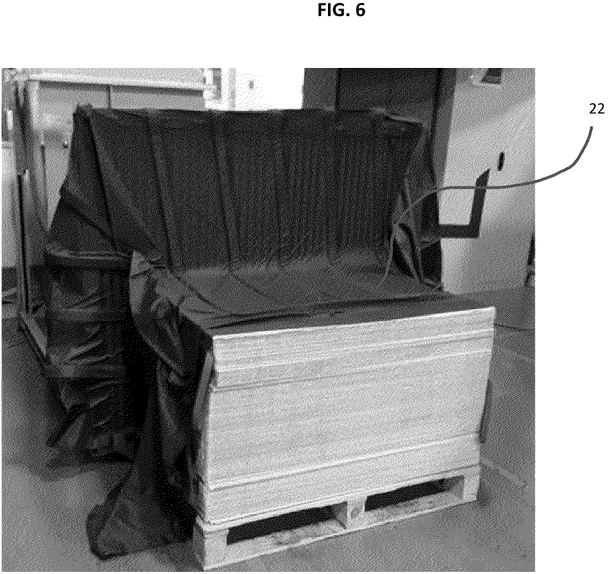

To assess the effect of using a flexible shroud to cause air to flow primarily through the flutes of the cardboard of the cardboard stack, comparisons were conducted: stacks of corrugated cardboard sheets that had been cut to the same size (1050 mm × 750 mm) with a laser cutter were subjected to airflow in an open configuration similar to that shown in FIG. 2, with and without the use of a shroud. In the case without the shroud, the stack was arranged to so that one end of the stack approximately fit the hard-edged opening of the airflow device, but there were gaps around the edges of that opening. The actual configuration used is shown in FIG. 5, in which the stack of cardboard sheets 10 is piled on a pallet 60, and aligned so that one end of the stack (that opposite the viewer) is within the aperture that is created by the bottom of the opening in the apparatus (which is the same height as the pallet) and sliding shutters 62, 64 and 66; the gap along the top of the stack is indicated by 68, and there are also gaps (not seen) along the vertical edges of the stack. When the shroud was used, the gaps were covered. FIG. 6 shows the actual configuration used when the shroud was employed. Airflow speed was measured by placing an airflow measurement device (MRC Anemometer catalog no. AM-4207SD, www.mrclab.com/anemometertype-kj-temp-data-logger-1), at the front center the stack, directly in front of the end where the air flowed into the stack. Effectiveness in removing the odor that remained from the laser cutting was assessed by the Robinson method (similar to European standard EN 1230-2): each of seven trained panelists evaluated the level of odor by putting the laser-cut portion of the topmost sheet close to his nose and rating the smoke smell on a scale of 1-10, with 1 being the smell of uncut carton and 10 being the smell of the carton immediately after being cut with a laser. “Extraction time” was the time it took for the average odor rating to be reduced to 2 under the same airflow speed. Results are presented in Table 1 below. Assessments were done for both B flute and E flute type cardboard.

TABLE 1

| Flute type | Sheets in pallet | Cover type | Airflow m/s | Extraction time, min. |
|---|---|---|---|---|
| B | 270 | Hard | 3.2 | 40 |
| B | 270 | Flexible | 6.0 | 15 |
| E | 460 | Hard | 0.7 | 50 |
| E | 460 | Flexible | 2.7 | 30 |

These results show that the use of a flexible shroud significantly increases the airflow through the flutes and reduces the time required to remove the odor that results from laser cutting the corrugated cardboard.

1. A method of conditioning a corrugated cardboard stack, which comprises causing air to flow through flutes of the stack from an air intake end to an air discharge end of the stack by confining air moved by an air moving device to flow only, or at least predominantly, through the flutes.

2. A method as claimed in claim 1, wherein the air moving device includes an air extractor connected to the air discharge end of the stack to place the air discharge end under sub-atmospheric pressure and thereby to draw ambient air into the flutes at the air intake end.

3. A method as claimed in claim 1 or 2, wherein the air moving device includes a fan or blower connected to the air intake end of the stack to raise the air pressure at the air intake end above ambient atmospheric pressure and thereby blow air into the flutes.

4. A method according to any one of claims 1 to 3, wherein the air moving device includes a flexible shroud that surrounds the stack so as to confine air moved by the device to flow only, or at least predominantly, through flutes of the cardboard in the stack, from the air intake end to the air discharge end of the stack.

5. A method as claimed in any one of claims 2 to 4, wherein the same air moving device serves both to reduce the air pressure at the discharge end of the stack and to increase the air pressure at the air intake end, thereby causing air to recirculate through the stack.

6. A method as claimed in claim 5, wherein the recirculated air is filtered.

7. A method as claimed in claim 5 or 6, wherein the temperature of the recirculating air is controlled by heating and/or cooling to remain within a predetermined range.

8. A method according to claim 7 wherein said predetermined range is from 30° C. and 80° C.

9. A method according to claim 8 wherein said predetermined range is from 40° C. to 60° C.

10. A method as claimed in claim 5, 6, 7, 8 or 9, wherein the humidity of the recirculating air is controlled by a humidifier to remain within a predetermined range.

11. A method according to claim 10 wherein said predetermined range is from 40% to 80%.

12. The method of claim 11, wherein said predetermined range is from 50% to 70%.

13. A method as claimed in any one of claims 5 to 12, wherein ozone or other airborne material is introduced into the recirculating air.

14. A method as claimed in any one of claims 1 to 13, wherein the speed of the air in the flutes in the range from 0.3 m/s to 15 m/s.

15. A method as claimed in any one of claims 1 to 14, wherein the speed of the air in the flutes in the range from 2.5 m/s to 10 m/s.

16. A method as claimed in any one of claims 1 to 15, wherein the corrugated cardboard stack is a stack of corrugated cardboard that has been cut with a laser cutter.

17. A method according to claim 16, wherein prior to said commencement of causing air to flow through flutes of the stack the cardboard has an unpleasant odor as a result of said cutting with a laser cutter, and the flow of air through the flutes of the stack is continued for a time sufficient to reduce said odor to an acceptable level.

18. An apparatus for conditioning a corrugated cardboard stack, which comprises an air moving device and a shroud to surround the stack so as to confine air moved by the device to flow only, or at least predominantly, through flutes of the cardboard in the stack, from an air intake end to an air discharge end of the stack.

19. An apparatus as claimed in claim 18, wherein the shroud includes an element made of a flexible material that is urged to seal against the sides of the stack by an air pressure differential generated by the air moving device.

20. An apparatus as claimed in claim 18 or 19, wherein the air moving device comprises an air extractor connected to the air discharge end of the stack to place the air discharge end under sub-atmospheric pressure and thereby to draw ambient air into the flutes at the air intake end.

21. An apparatus as claimed in any one of claims 18 to 20, wherein the air moving device comprises a fan or blower connected to the air intake end of the stack to raise the air pressure at the air intake end above ambient atmospheric pressure and thereby blow air into the flutes.

22. An apparatus as claimed in claims 20 and 21, wherein the same air moving device serves both to reduce the air pressure at the discharge end of the stack and to increase the air pressure at the air intake end, thereby creating an air recirculation circuit passing through the flutes of the stack.

23. An apparatus as claimed in claim 22, wherein the recirculation circuit includes a filter.

24. An apparatus as claimed in claim 22 or 23, wherein the recirculation circuit includes at least one of
(i) a heater or cooler for maintaining the temperature of the recirculating air within a predetermined range,
(ii) a humidifier for maintaining the humidity of the recirculating air within a predetermined range,
(iii) an ozoniser for introducing ozone into the recirculating air, and
(iv) and atomiser for introducing airborne material into the recirculating air.

25. An apparatus as claimed in claim 24, further comprising a temperature sensor and a controller for controlling the heater or cooler to maintain the circulating air within the range from 30° C. to 80° C.

26. An apparatus as claimed in claim 25, the controller maintains the circulating air within the range from 40° C. to 60° C.

27. An apparatus as claimed in claim 24 or 26, further comprising a humidity sensor and the control is operative to maintain the relative humidity of circulating air in the range from 40%-to 80%.

28. An apparatus as claimed in claim 27, wherein the control is operative to maintain the relative humidity of circulating air in the range from 50% to 70%.

29. An apparatus as claimed in any one of claims 25 to 28, wherein the controller is operative to control the air moving device to maintain the speed of the air in the flutes in the range from 0.3 m/s to 15 m/s.

30. An apparatus claimed in claim 29, wherein the controller is operative to control the air moving device to maintain the speed of the air in the flutes in the range from 2.5 m/s to 10 m/s.

31. An apparatus as claimed in any one of claims 18 to 30, further comprising a corrugated cardboard stack in which said corrugated has been cut with a laser cutter.

32. An apparatus according to claim 31, wherein the cardboard has an unpleasant odor as a result of said cutting with a laser cutter.
